(19) United States
(12) Patent Application Publication
(10) Pub. No.: US 2009/0069837 A1
(43) Pub. Date: Mar. 12, 2009

(54) HAND AND INSTRUMENT ACCESS DEVICE

(75) Inventors: Frank BONADIO, Bray (IE); Trevor VAUGH, Birr (IE); John BUTLER, Blackrock (IE); Shane J. MACNALLY, Delgany (IE)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: ATROPOS LIMITED

(21) Appl. No.: 12/194,907

(22) Filed: Aug. 20, 2008

Related U.S. Application Data
(60) Provisional application No. 60/935,570, filed on Aug. 20, 2007.

(51) Int. Cl. A61M 29/00

(52) U.S. Cl. 606/191

(57) ABSTRACT

A surgical sealing device 1 for providing hand or instrument access during a surgical procedure is mounted to a wound retractor device 10 which is used to retract the sides of an opening into an internal cavity. The sealing device 1 comprises a first sealing member 5 and a second sealing member 6. The second sealing member 6 has passageway 2 with a sidewall of an elastic material to create a seal between the sidewall of the passageway 2 and an object passing through the passageway 2. The first sealing member 5 is in the form of a flap of flexible material and is moveable relative to the second sealing member 6 between a closed configuration sealing the passageway 2 and an open configuration. The flap member 5 may be fixedly attached to the second sealing member 6 by means of a reception loop 7 formed at the distal surface of the second sealing member 6.

This invention relates to a surgical sealing device.

### STATEMENTS OF INVENTION

According to the invention there is provided a surgical sealing device comprising:
a sealing valve for sealing across an opening to an internal cavity;
the sealing valve comprising a first sealing member and a second sealing member;
the sealing members being movable relative to one another between a closed configuration in which the sealing members at least partially overlap one another for sealing across the opening, and an open configuration for facilitating passage of an object through the sealing valve to access the internal cavity.

In a preferred embodiment the first sealing member is attached to the second sealing member. The device may comprise a holder to hold the first sealing member attached to the second sealing member. The holder may comprise a hinge. The holder may comprise one or more fixing elements.

In one embodiment at least a portion of the first sealing member may be formed integrally with the second sealing member.

In another case the device comprises a connector member for connecting the sealing valve to a retractor device. The connector member may be configured to extend substantially distally from the sealing valve to a retractor device. The connector member may be of a flexible material. The connector member may comprise a sleeve.

In one embodiment the second sealing member comprises an access opening extending therethrough. The second sealing member may comprise a lipseal. In the closed configuration, the first sealing member may extend across the access opening to seal the access opening. The access opening may be sized to seal with a surgeons forearm in one case or an instrument shaft in another case.

In one embodiment of the invention the first sealing member is of a flexible material. The first sealing member may be of a silicone material. The first sealing member may be of a TPE material. The first sealing member may be of a fabric material.

In one case the second sealing member is at least partially of a gelatinous elastomeric material. The material may comprise a plasticiser. The plasticiser may be selected from the group consisting of naturally derived oils, synthetic oils and liquid oligomers. The second sealing member may be at least partially of a TPE material. The second sealing member may be at least partially of a silicone material.

In another embodiment the first sealing member is biased towards the closed configuration. The second sealing member may be substantially curved. A concave portion of the curved second sealing member may face distally. In the closed configuration, the first sealing member may be substantially curved. In the closed configuration, a concave portion of the curved first sealing member may face distally.

In another aspect of the invention there is provided a surgical assembly comprising:
a retractor device for retracting the sides of an opening to an internal cavity; and
a surgical sealing device of the invention.

In one embodiment of the invention the surgical sealing device is mounted to the retractor device. The sealing valve of the surgical sealing device may be located adjacent to a proximal end of the retractor device. The sealing valve of the surgical sealing device may be spaced-apart from the retractor device. The sealing valve may be located proximally of the retractor device.

### BRIEF DESCRIPTION OF THE DRAWINGS

The invention will be more clearly understood from the following description of some embodiments thereof, given by way of example only, with reference to the accompanying drawings, in which:—

FIG. 1 is a partially cross-sectional, side view of a surgical sealing device according to the invention;

FIG. 2 is a cross-sectional, side view of part of the device of FIG. 1;

FIG. 3 is a plan view from below of the part of FIG. 2;

FIG. 4 is an isometric view from below of the part of FIG. 2;

FIG. 5 is an isometric view from above of the part of FIG. 2;

FIG. 6 is a plan view of a mounting detail of the device of FIGS. 1 to 5;

FIG. 7 is an isometric, partially cut-away view of the detail of FIG. 6;

FIG. 8 is a view similar to FIG. 2 of part of another surgical sealing device according to the invention;

FIG. 9 is a view similar to FIG. 2 of part of another surgical sealing device according to the invention;

FIGS. 10 and 11 are views similar to FIGS. 3 and 4 of part of another surgical sealing device according to the invention;

FIG. 12 is a view similar to FIG. 3 of part of another surgical sealing device according to the invention;

FIG. 13 is an isometric view of a sealing member of the part of FIG. 12;

FIGS. 16 and 17 are views similar to FIG. 1 of another surgical sealing device according to the invention, in use;

FIGS. 18 to 20 are views similar to FIG. 1 of a further surgical sealing device according to the invention, in use;

FIG. 21 is a cross sectional view of another surgical sealing device according to the invention; and

FIG. 22 is a cross sectional view of the device of FIG. 21, in use.

### DETAILED DESCRIPTION

Referring to the drawings and initially to FIGS. 1 to 5 thereof, there is illustrated a surgical sealing device 1 according to the invention, for providing hand or instrument access during a surgical procedure, such as a laparoscopic procedure or a hand-assisted laparoscopic procedure.

The sealing device 1 may be mounted to a wound retractor device 10 as illustrated in FIG. 1. The retractor device 10 is employed to retract the sides of an opening to an internal cavity. The retractor may be of any suitable construction such as the retractors described in US 2001/00370853A and U.S. Pat. No. 6,582,364, and/or US 2005/0090717A, the whole contents of which are incorporated herein by reference.

The device 1 may in the case of a laparoscopic procedure be employed to effect a seal around a laparoscopic instrument inserted through the device 1 into an abdomen to maintain insufflation gas pressure within the abdomen. In the case of a hand-assisted laparoscopic procedure, the device 1 may be employed to effect a seal around a surgeon's hand or forearm inserted through the device 1 into the abdomen to maintain insufflation gas pressure within the abdomen.

The device 1 comprises a first sealing member 5 and a second sealing member 6.

The second sealing member 6 has a passageway 2 extending therethrough which an object, such as a laparoscopic instrument or a surgeon's hand/forearm 3, may be inserted to access an interior space, such as an insufflated abdomen 4. The sidewall of the passageway 2 is of an elastic material, and the passageway 2 typically has a smaller diameter than an object to be inserted therethrough. The passageway 2 thus creates a seal between the sidewall of the passageway 2 and the object, defining an interference fit between the sidewall and the object (FIG. 1). In this manner, the passageway 2 through the second sealing member 6 acts as a lipseal valve to seal around the object while the object is inserted through the passageway 2 and into the abdomen 4. Thus leakage of insufflation gas out of the abdomen 4 while the object is inserted through the passageway 2 and into the abdomen 4 is prevented.

In the case of hand-assisted laparoscopic surgery, the passageway 2 is sized to seal around the hand/forearm 3 of the surgeon.

The second sealing member 6 may be of a gelatinous elastomeric material. The material may include a plasticiser which may be selected from the group consisting of naturally derived oils, synthetic oils and liquid oligomers. Alternatively the second sealing member 6 may be of TPE, or silicone.

The gel material may include an elastomer, such as silicone or latex. The gel material may also include an oil, and/or a foam.

In one embodiment the second sealing member 6 is of a gelatinous elastomeric material. An extensive review of gelatinous elastomeric materials is included in U.S. Pat. No. 5,994,450 (Pierce), the entire contents of which are incorporated herein by reference. One such group of gelatinous elastomers may comprise a triblock copolymer A-B-A wherein A is selected from the group consisting of monoalkenylarene polymers and B is a hydrogenated polymer including a plurality of isoprene monomers and a plurality of butadiene monomers. The material includes a plasticiser which may be selected from the group consisting of naturally derived oils, synthetic oils and liquid oligomers. For the device of this embodiment of the invention the gelatinous elastomeric material is formulated to have high tear strength and high flexibility.

The materials required to form a suitable gel material are available, for example, from the company Edizone L.C. of Utah, USA. The gel material preferably has the properties of high tear strength to resist tearing and high flexibility for passage of an object through the passageway 2 into the abdomen 4.

The first sealing member 5 may be employed to seal the passageway 2. The first sealing member 5 is provided in the form of a flap member of flexible material. The first sealing member 5 may be of silicone, or TPE, or a fabric, or polyurethane, or any other suitable material. The first sealing member 5 may be of a more rigid material or a film-like material.

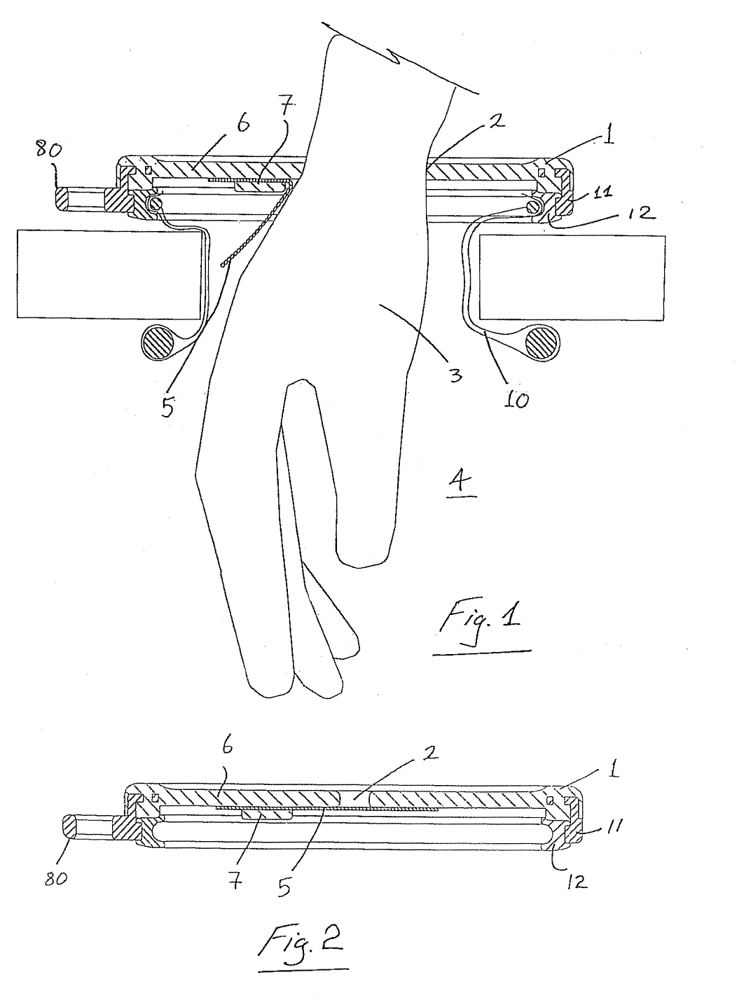

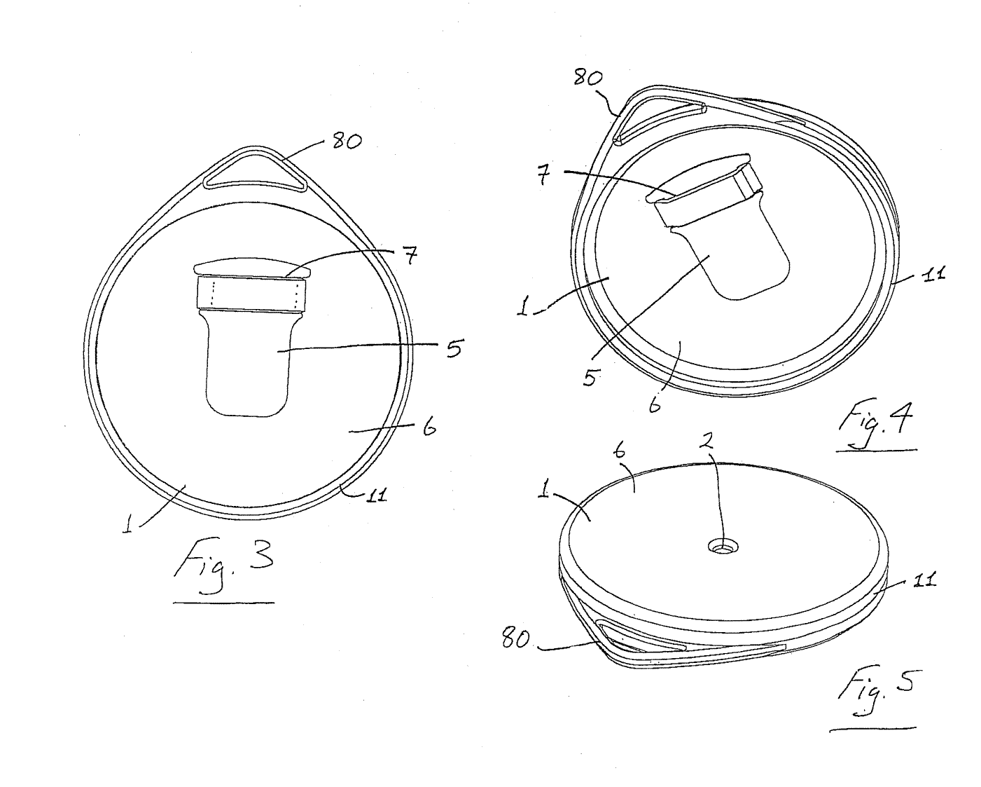

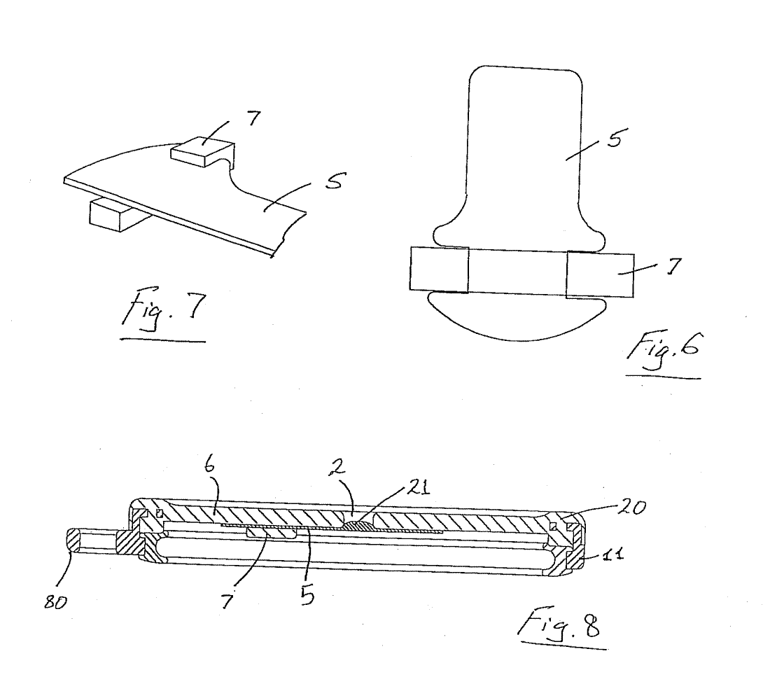

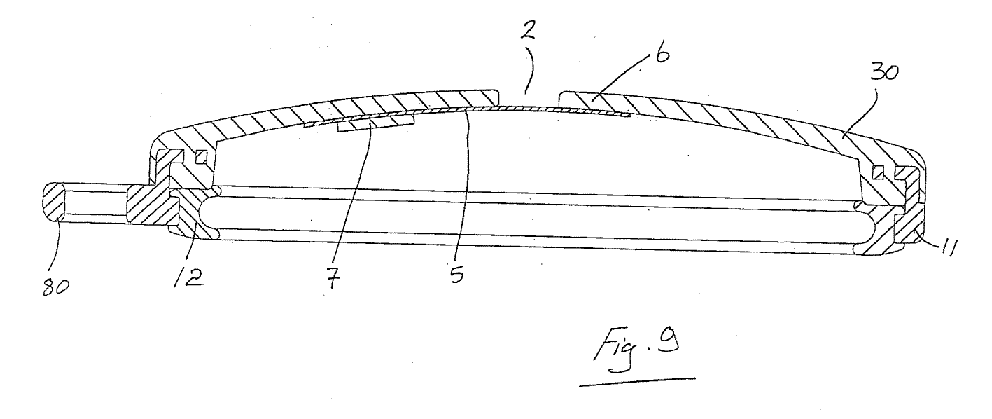

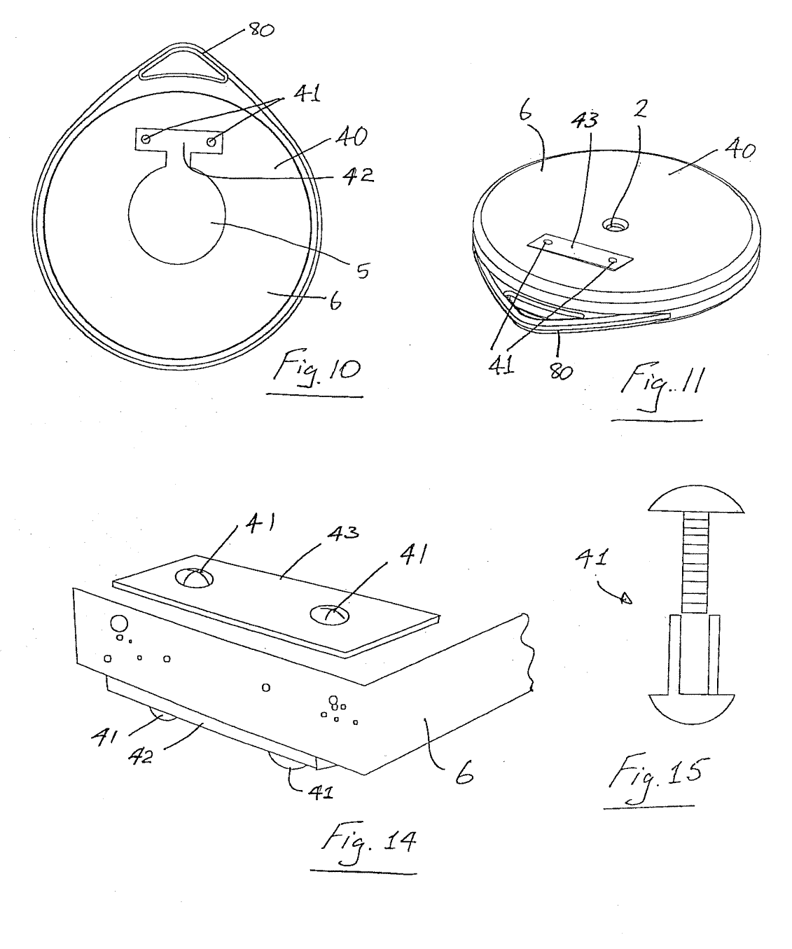

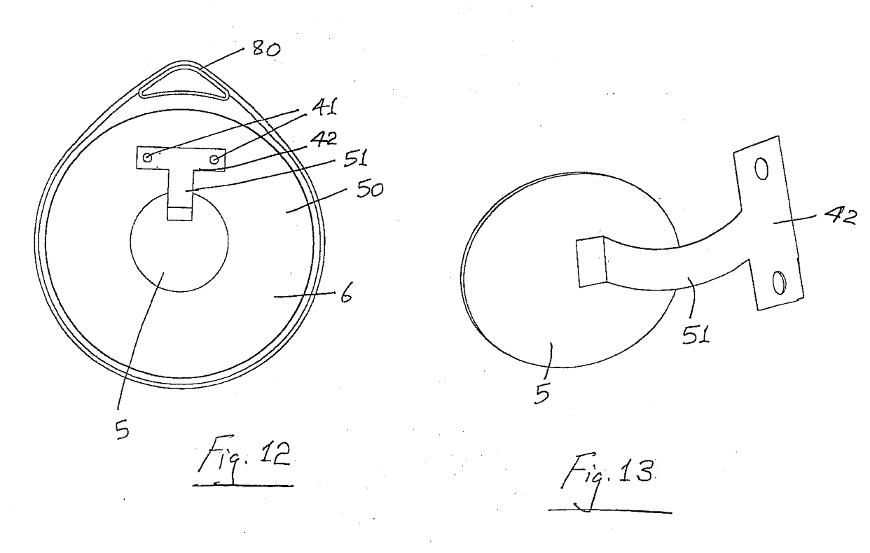

The flap member 5 is movable relative to the second sealing member 6 between a closed configuration sealing the passageway 2 (FIG. 2), and an open configuration to facilitate insertion of an object, such as the surgeon's hand/forearm 3 through the passageway 2 to access the abdomen 4 (FIG. 1).

In the closed configuration, the flap member 5 extends across the distal end of the passageway 2 and overlaps the entire periphery of the distal end of the passageway 2 to prevent leakage of the insufflation gas out of the abdomen 4. The insufflation gas pressure within the abdomen 4 forces the flap member 5 into engagement with the second sealing member 6 in the closed configuration. Because the first sealing member 5 and the second sealing member 6 overlap in the closed configuration, a seal is created across the wound opening which prevents loss of insufflation gasses from the abdomen 4. The overlapping first and second sealing members 5, 6 thus provide a sealing valve for sealing across the wound opening.

In the open configuration, the flap member 5 is retracted to reveal the distal end of the passageway 2 to enable an object, such as the surgeon's hand/forearm 3 to pass through the sealing device 1 and into the abdomen 4. In this manner the flap member 5 defines an overlap seal. Insertion of the object, such as the hand/forearm 3 through the passageway 2 pushes against the flap member 5 to cause retraction of the flap member 5.

The first sealing member 5 is formed separately from the second sealing member 6. The first sealing member 5 is fixedly attached to the second sealing member 6 by means of extending the first sealing member 5 through a reception loop 7 formed at the distal surface of the second sealing member 6 (FIGS. 3 and 4). The reception loop 7 acts as a hinge holder to hold the first sealing member 5 attached to the second sealing member 6, and to enable the first sealing member 5 to move relative to the second sealing member 6 in a hinging action.

When the surgical sealing device 1 is mounted to the wound retractor device 10, the sealing members 5, 6 are adjacent to the proximal end of the wound retractor device 10.

FIG. 1 illustrates that when the surgeon puts his hand 3 through the lipseal 2, the flap member defines a trapdoor 5 which falls out of the way. When he removes his hand 3 the gas pressure and the hinge bias of the trapdoor 5 forces the trapdoor 5 back up to cover the hole 2.

FIG. 2 illustrates the trapdoor/flap 5 sealing the lipseal hole 2.

FIG. 3 shows the underside of the hand access valve 1. The valve 1 comprises the lipseal valve 2 made from gel/TPE/silicone. The valve 2 is then overmoulded onto a ring 11. The ring 11 incorporates the connection means to a mounting ring 12 of the retractor 10. The ring 11 also has a protruding part 80 to facilitate ease of gripping for removal of the ring 11 from the retractor 10.

FIG. 4 shows the trapdoor/flap 5 which is made from either silicone/TPE/textile. The trapdoor 5 is held in place by the raised section of the reception loop 7 of gel/TPE/silicone. The loop 7 is integrally formed with the remainder of the second sealing member 6. The trapdoor 5 sits underneath the raised section 7 as illustrated particularly in FIGS. 6 and 7. FIG. 3 illustrates the removal loop 80, the overmould ring 11, and the gel/TPE/silicone. FIG. 4 illustrates the raised section 7, and the trapdoor/flap 5. FIG. 5 illustrates the lipseal 2.

In FIG. 8 there is illustrated another surgical sealing device 20 according to the invention, which is similar to the surgical sealing device 1 of FIGS. 1 to 5, and similar elements in FIG. 8 are assigned the same reference numerals.

In this case the first sealing member 5 comprises a protrusion or bump 21 at the proximal surface of the first sealing member 5. In the closed configuration, the protrusion 21 extends proximally into the passageway 2 to prevent leakage of insufflation gas from the abdomen through the passageway 2. FIG. 6 illustrates the trapdoor/flap 5 with the bump 21 which creates a greater seal.

FIG. 9 illustrates another surgical sealing device 30 according to the invention, which is similar to the surgical sealing device 1 of FIGS. 1 to 5, and similar elements in FIG. 9 are assigned the same reference numerals.

In this case the second sealing member 6 is curved with a concave portion of the curve facing distally. In the closed configuration, the first sealing member 5 is also curved with a concave portion of the curve facing distally. By arranging the sealing members 5, 6 in this curved manner, this causes the first sealing member 5 to be biased towards the closed configuration.

FIG. 9 shows the section view of the flap/trapdoor hand access valve 30. This version has the domed gel. The reason for the dome is to help force the flap/trapdoor 5 into the closed position. FIG. 9 illustrates the removal loop 80, the lipseal 2, the domed gel 6, the flap/trapdoor 5, the gel housing 11, and the retractor base 12.

Referring to FIGS. 10 and 11 there is illustrated another surgical sealing device 40 according to the invention, which is similar to the surgical sealing device 1 of FIGS. 1 to 5, and similar elements in FIGS. 10 and 11 are assigned the same reference numerals.

In this case the first sealing member 5 has a mounting part 42 which is fixedly attached to the second sealing member 6 by means of fixing pins or rivets 41 as illustrated particularly in FIGS. 14 and 15. A bearing plate 43 may also be provided on the proximal side of the second sealing member 6.

FIGS. 10 and 11 illustrate the gel lipseal 6, and the rivets 41.

In FIGS. 12 and 13 there is illustrated another surgical sealing device 50 according to the invention, which is similar to the surgical sealing device 40 of FIGS. 10 and 11, and similar elements in FIGS. 12 and 13 are assigned the same reference numerals.

In this case the first sealing member 5 is attached to the second sealing member 6 by means of a resilient hinge arm 51. The hinge arm 51 may be of a plastics material. The hinge arm 51 is attached to the second sealing member 6 by means of the fixing pins or rivets 41.

FIGS. 12 and 13 illustrate the circular plastic flap 5, and the plastic hinge 51.

FIGS. 16 and 17 illustrate another surgical sealing device 60 according to the invention, which is similar to the surgical sealing device 1 of FIGS. 1 to 5, and similar elements in FIGS. 16 and 17 are assigned the same reference numerals.

In this case the surgical sealing device 60 is connected to a wound retractor device 10 by means of a flexible sleeve 61. The sleeve 61 extends distally from the surgical sealing device 60 to the mounting ring 11 of the wound retractor device 10. The sealing members 5, 6 are spaced-apart from the wound retractor device 10, located proximally of the wound retractor device 10.

FIGS. 16 and 17 show the trapdoor hand access valve 60 spaced proximally from the retractor base 10. In this manner the hand access device 60 is offset from the retractor base 10. This offset arrangement, enables the hand access device 60 to move laterally relative to the retractor base 10. The offset may be created using a film material or an elasticated rubber. FIG. 16 illustrates the gel lipseal 6, the sleeve/rubber 61, and the retractor base 10. FIG. 17 illustrates the trapdoor/flap 5.

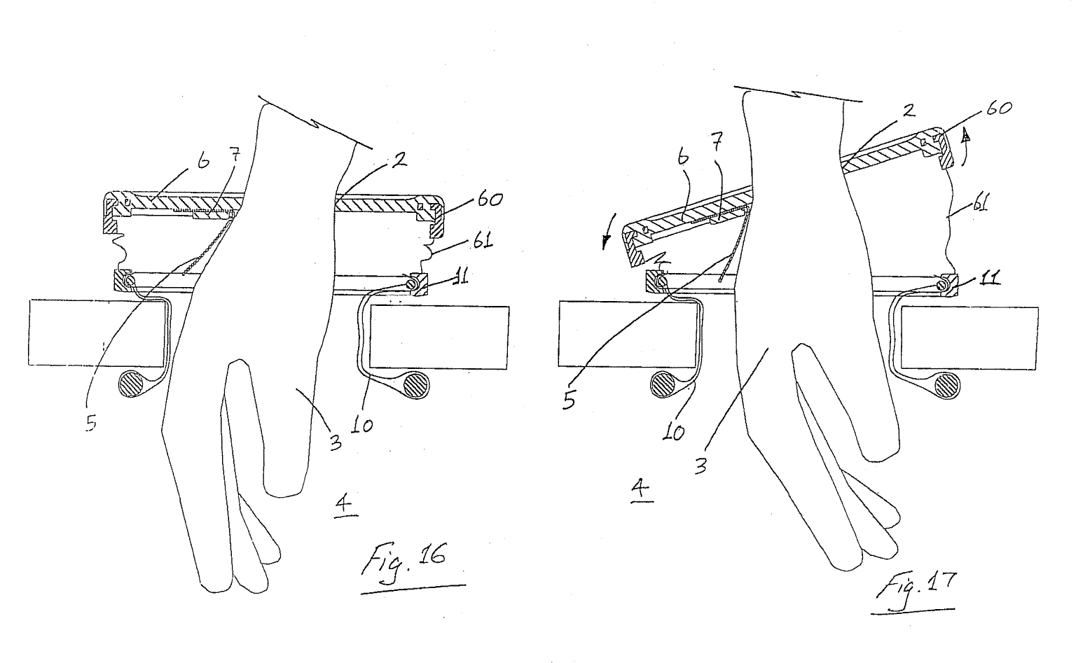

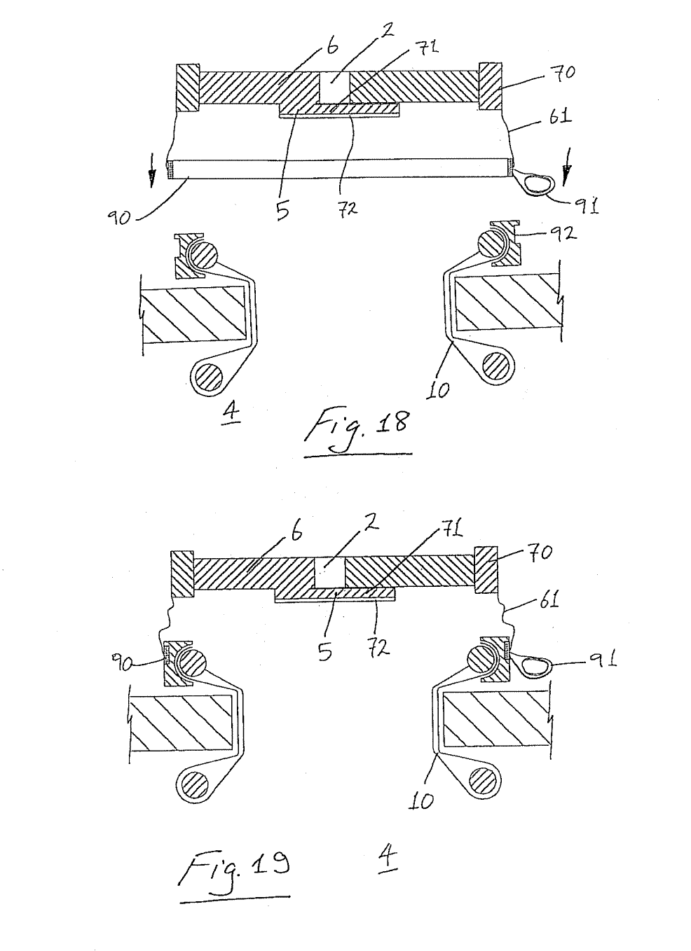

Referring to FIGS. 18 to 20 there is illustrated a further surgical sealing device 70 according to the invention, which is similar to the surgical sealing device 60 of FIGS. 16 and 17, and similar elements in FIGS. 18 to 20 are assigned the same reference numerals.

In this case the first sealing member 5 comprises a proximal portion 71 formed integrally with the second sealing member 6, and a distal portion 72 formed separately from the second sealing member 6. The distal portion 72 acts as a reinforcement for the proximal portion 71. The distal portion 72 may be of a stiffer material than the proximal portion 71. The stiffer distal portion 72 assists in reinforcing the more flexible proximal portion 71 to facilitate ease of passage of the hand/arm 3 through the passageway 2.

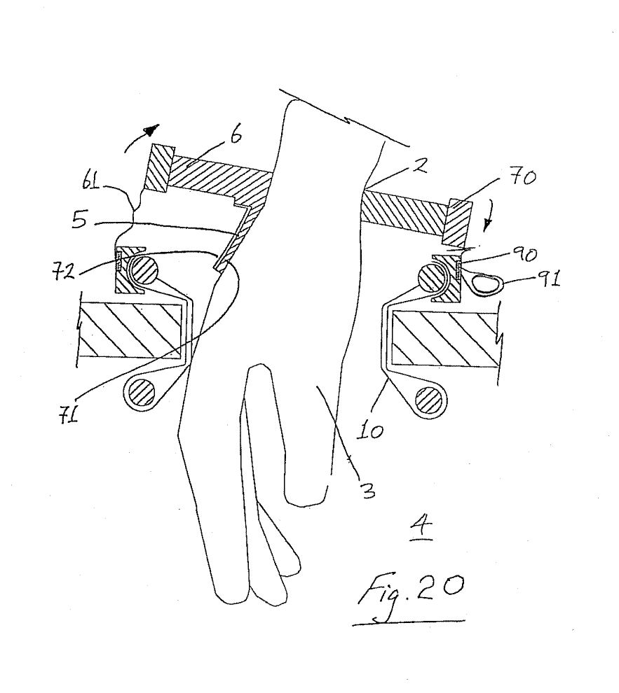

FIG. 18 illustrates an elasticated connection 90, a removal ring 91, and a retractor connection point 92. The elasticated connection 90 may be stretched around the connection point 92 to mount the surgical sealing device 70 to the wound retractor 10. The removal ring 91 provides a means of detaching the elasticated connection 90 from the wound retractor 10, if required. FIG. 19 illustrates the overlap valve 6. FIGS. 18 to 20 show the overlap valve 70 attached to the retracting base 10 via the flexible sleeve 61. The sleeve 61 allows the surgeon better movement and more comfort.

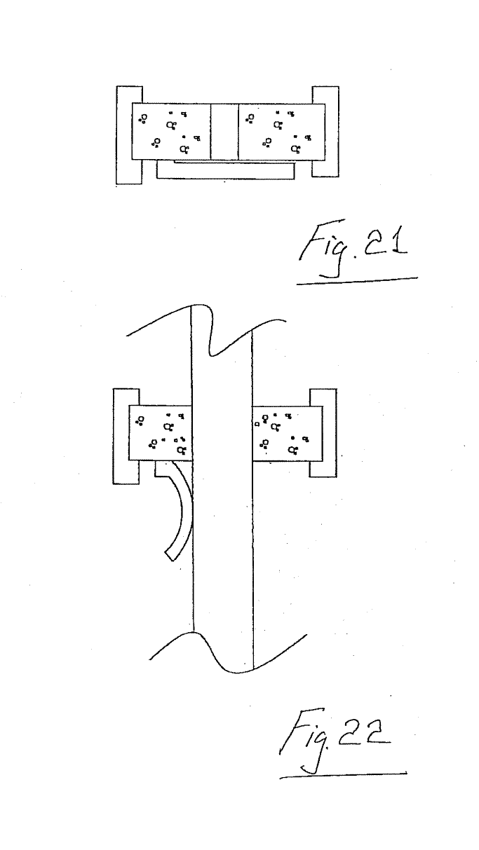

Referring to FIGS. 21 and 22 there is illustrated another sealing device 95 according to the invention. The sealing device 95 is similar to that described above with reference to FIGS. 1 to 5 and like parts are assigned the same reference numerals. In this case the device 95 is particularly suitable for receiving an instrument 96. It will be appreciated that any of the devices described herein may be adapted for use with a surgical instrument.

The invention is not limited to the embodiments hereinbefore described, with reference to the accompanying drawings, which may be varied in construction and detail.

1. A surgical sealing device comprising:
a sealing valve for sealing across an opening to an internal cavity;
the sealing valve comprising a first sealing member and a second sealing member;
the sealing members being movable relative to one another between a closed configuration in which the sealing members at least partially overlap one another for sealing across the opening, and an open configuration for facilitating passage of an object through the sealing valve to access the internal cavity,
the first sealing member being attached to the second sealing member.

2. A device as claimed in claim 1 wherein the device comprises a holder to hold the first sealing member attached to the second sealing member.

3. A device as claimed in claim 2 wherein the holder comprises a hinge.

4. A device as claimed in claim 2 wherein the holder comprises one or more fixing elements.

5. A device as claimed in claim 1 wherein the second sealing member comprises an access opening extending therethrough.

6. A device as claimed in claim 5 wherein the access opening is sized to seal with a surgeons forearm.

7. A device as claimed in claim 6 wherein the access opening is sized to seal with an instrument shaft.

8. A device as claimed in claim 5 wherein the second sealing member comprises a lipseal.

9. A device as claimed in claim 5 wherein in the closed configuration, the first sealing member extends across the access opening to seal the access opening.

10. A device as claimed in claim 1 wherein the device comprises a connector member for connecting the sealing valve to a retractor device.

11. A device as claimed in claim 10 wherein the connector member is configured to extend substantially distally from the sealing valve to a retractor device.

12. A device as claimed in claim 10 wherein the connector member is of a flexible material.

13. A device as claimed in claim 10 wherein the connector member comprises a sleeve.

14. A device as claimed in claim 1 wherein the first sealing member is of a flexible material.

15. A device as claimed in claim 1 wherein the second sealing member is at least partially of a gelatinous elastomeric material.

16. A device as claimed in claim 1 wherein the second sealing member is at least partially of a TPE material.

17. A device as claimed in claim 1 wherein the second sealing member is at least partially of a silicone material.

18. A device as claimed in claim 1 wherein the first sealing member is biased towards the closed configuration.

19. A device as claimed in claim 1 wherein the second sealing member is substantially curved.

20. A device as claimed in claim 19 wherein a concave portion of the curved second sealing member faces distally.

21. A device as claimed in claim 18 wherein in the closed configuration, the first sealing member is substantially curved.

22. A device as claimed in claim 21 wherein in the closed configuration, a concave portion of the curved first sealing member faces distally.

23. A surgical assembly comprising:
a retractor device for retracting the sides of an opening to an internal cavity; and
a surgical sealing device as claimed in claim 1.

24. An assembly as claimed in claim 23 wherein the surgical sealing device is mounted to the retractor device.

25. An assembly as claimed in claim 23 wherein the sealing valve of the surgical sealing device is located adjacent to a proximal end of the retractor device.

26. An assembly as claimed in claim 23 wherein the sealing valve of the surgical sealing device is spaced-apart from the retractor device.

27. An assembly as claimed in claim 26 wherein the sealing valve is located proximally of the retractor device.
